(19) United States
(12) Patent Application Publication
(10) Pub. No.: US 2026/0072043 A1
(43) Pub. Date: Mar. 12, 2026

(54) BIOMARKERS FOR DETECTING AND/OR DETERMINING A TREATMENT REGIMEN FOR ALZHEIMER'S DISEASE

(71) Applicant: Seq Biomarque, LLC, Anaheim, CA (US)

(72) Inventors: Sarah BACUS, Hinsdale, IL (US); Christopher HAMM, Chicago, IL (US); Jeff OLSON, Yorba Linda, CA (US)

(21) Appl. No.: 19/107,477

(22) PCT Filed: Aug. 28, 2023
(86) PCT No.: PCT/US2023/031296

§ 371 Date: Feb. 27, 2025

Related U.S. Application Data
(60) Provisional application No. 63/401,947, filed on Aug. 29, 2022.

(51) Int. Cl. G01N 33/68

(52) U.S. Cl. CPC G01N 33/6896; G01N 33/6872; G01N 2440/14; G01N 2800/2821

(57) ABSTRACT

The present disclosure provides methods for diagnosing or determining susceptibility to Alzheimer's Disease a subject by obtaining a biological sample from the subject; detecting one or more biomarkers in the biological sample selected from the group consisting of: inflammation biomarkers, oxidative stress biomarkers, insulin resistance biomarkers, and autophagy biomarkers; and diagnosing the subject with Alzheimer's Disease where one or more of the biomarkers is detected in the biological sample. Also provided are methods of treating a subject with Alzheimer's Disease comprising administering to the subject an effective amount of one or more agents for the treatment of inflammation, oxidative stress, insulin resistance, and/or autophagy.

### FIELD

The present disclosure provides methods for diagnosing or determining susceptibility to Alzheimer's Disease in a subject and optionally subsequent treatment of the subject including, for example, by administering to the subject an effective amount of one or more agents for the treatment of inflammation, oxidative stress, insulin resistance, and/or autophagy.

### BACKGROUND

Alzheimer's disease (AD) is a neurodegenerative disease of undefined etiology and limited diagnostic markers. Clinicopathological studies suggest that AD pathology (particularly the buildup of amyloid plaques) begins 10-20 years before cognitive symptoms. However, a definitive diagnosis of AD can still only be obtained via neuropathologic evaluation at autopsy. Data suggest an early and insidious pathogenesis of AD, the clinical manifestation of which becomes apparent only after substantial neuronal cell death and synapse loss has taken place. However, current treatment modalities are limited by imperfect diagnostic parameters and a complete inability to identify the early pathogenic disease process.

Early diagnosis is an essential step in the treatment of Alzheimer's Disease, as early diagnosis allows subjects to receive drugs that may slow disease progression. However, an accurate diagnosis can be difficult, especially for patients having mild or early-stage Alzheimer's Disease. An unambiguous diagnosis may be made by examining the pathology of brain tissue, but this is only feasible posthumously.

Accordingly, a need exists for methods to detect biomarkers associated with Alzheimer's disease and the use of such biomarkers in the treatment of Alzheimer's Disease.

### SUMMARY

The present disclosure provides methods of treating a subject with Alzheimer's Disease, or treating a subject susceptible to developing Alzheimer's Disease (e. g, a subject having one or more inflammation biomarkers, oxidative stress biomarkers, insulin resistance biomarkers, and/or autophagy biomarkers), by administering to the subject a therapeutically effective amount of one or more agents for the treatment of inflammation, oxidative stress, insulin resistance, and/or autophagy.

In some embodiments, the subject is administered one or more agents for the treatment of inflammation. In some embodiments, the agent is rapamycin, minocycline, thalidomide or its analogues, rosiglitazone, an NSAID, or pioglitozone. In some embodiments, the NSAID is naproxen or rofecoxib.

In some embodiments, the subject is administered one or more agents for the treatment of oxidative stress. In some embodiments, the agent is hydrogen peroxide.

In some embodiments, the subject is administered one or more agents for the treatment of insulin resistance. In some embodiments, the agent is insulin, thiazolidinediones, an incretin receptor agonist (IRAs), glucagon like peptide 1 (GLP-1), glucagon like peptide 2 (GLP-2), or a peroxisome proliferator-activated receptor gamma (PPARγ) agonist. In some embodiments, the IRA is liraglutide. In some embodiments, the PPARγ agonist is rosiglitazone.

In some embodiments, the subject is administered one or more agents for the treatment of autophagy. In some embodiments, the agent inhibits PIK3. In some embodiments, the agent is rapamycin, everolimus, temsirolimus, metformin, trehalose, curcumin or analogues thereof, aspirin, gemfibrozil, Wy14643, cinnamic acid, gypenoside XVII, ouabain, fisetin, or flubendazole.

The present disclosure also provides a method of treating a subject with, or suspected of having Alzheimer's disease, the method comprising: obtaining a biological sample from the subject with or suspected of having Alzheimer's disease; detecting one or more biomarkers in the biological sample selected from the group consisting of: inflammation biomarkers, oxidative stress biomarkers, insulin resistance biomarkers, and autophagy biomarkers; and administering to the subject a therapeutically effective amount of one or more agents that treat inflammation where one or more inflammation biomarkers are detected in the biological sample, one or more agents that treat oxidative stress where one or more oxidative stress biomarkers are detected in the biological sample, one or more agents that treat insulin resistance where one or more insulin resistance biomarkers are detected in the biological sample, and/or one or more agents that treat autophagy where one or more autophagy biomarkers are detected in the biological sample.

In some embodiments, the biological sample is cerebral spinal fluid (CSF). In some embodiments, the biological sample is blood.

In some embodiments, the inflammation biomarker is p38 MAPK, C-reactive protein (CRP), IL6, or TNFα. In some embodiments, the insulin resistance biomarker is insulin receptor substrate 1 (IRS-1). In some embodiments, the IRS-1 is phosphorylated at Ser616, Ser636, and/or Ser312.

In some embodiments, the autophagy biomarker is p62.

In some embodiments, the oxidative stress biomarker for is DNA damage, cleaved caspase 3, gamma-H2AX, BAD, or BAX.

In some embodiments, the subject is determined to have Alzheimer's disease where the one or more of the biomarkers detected in the biological sample are present in an amount at or above a threshold.

In some embodiments, the threshold is the amount of the one or more biomarkers in a biological sample obtained from a subject without Alzheimer's disease.

In some embodiments, the subject is administered one or more agents for the treatment of inflammation. In some embodiments, the agent is rapamycin, minocycline, thalidomide or its analogues, rosiglitazone, an NSAID, or pioglitozone. In some embodiments, the NSAID is naproxen or rofecoxib.

In some embodiments, the subject is administered one or more agents for the treatment of oxidative stress. In some embodiments, the agent is hydrogen peroxide.

In some embodiments, the subject is administered one or more agents for the treatment of insulin resistance. In some embodiments, the agent is insulin, thiazolidinediones, an incretin receptor agonist (IRAs), glucagon like peptide 1 (GLP-1), glucagon like peptide 2 (GLP-2), or a peroxisome proliferator-activated receptor gamma (PPARγ) agonist. In some embodiments, the IRA is liraglutide. In some embodiments, the PPARγ agonist is rosiglitazone.

In some embodiments, the subject is administered one or more agents for the treatment of autophagy. In some embodiments, the agent inhibits PIK3. In some embodiments, the agent is rapamycin, everolimus, temsirolimus, metformin, trehalose, curcumin or analogues thereof, aspirin, gemfibrozil, Wy 14643, cinnamic acid, gypenoside XVII, ouabain, fisetin, or flubendazole.

The present disclosure also provides a method of treating a subject with or suspected of having Alzheimer's disease, the method comprising: obtaining a biological sample from the subject with or suspected of having Alzheimer's disease; detecting one or more insulin resistance biomarkers; and administering to the subject an effective amount of one or more agents that treat insulin resistance where one or more insulin resistance biomarkers are detected in the biological sample.

The present disclosure also provides a method for diagnosing or determining susceptibility to Alzheimer's Disease in a subject in need thereof, the method comprising: obtaining a biological sample from the subject; detecting one or more biomarkers in the biological sample selected from the group consisting of: inflammation biomarkers, oxidative stress biomarkers, insulin resistance biomarkers, and/or autophagy biomarkers; and diagnosing the subject with Alzheimer's Disease, or determining that the subject is susceptible to developing Alzheimer's Disease, where one or more of the biomarkers are detected in the biological sample.

In some embodiments, the biological sample is cerebral spinal fluid (CSF). In some embodiments, the biological sample is blood.

In some embodiments, the inflammation biomarker is p38 MAPK. In some embodiments, the insulin resistance biomarker is insulin receptor substrate 1 (IRS-1). In some embodiments, the IRS-1 is phosphorylated at Ser616. In some embodiments, the IRS-1 is phosphorylated at Ser636. In some embodiments, the IRS-1 is phosphorylated at Ser616 and Ser636.

In some embodiments, the autophagy biomarker is p62.

In some embodiments, the oxidative stress biomarker for is DNA damage, cleaved caspase 3, gamma-H2AX, BAD, or BAX.

The present disclosure also provides a method for diagnosing Alzheimer's Disease or determining susceptibility to Alzheimer's Disease in a subject in need thereof, the method comprising: obtaining a biological sample (e.g., CSF) from the subject; detecting one or more insulin resistance biomarkers in the biological sample (e.g., phosphorylated Ser312, phosphorylated Ser616, and/or phosphorylated Ser636); and diagnosing the subject with Alzheimer's Disease, or determining that the subject is susceptible to developing Alzheimer's Disease, where one or more of insulin resistance biomarkers are detected in the biological sample. In a further embodiment, the subject is treated with one or more agents used in the treatment of insulin resistance including, for example, insulin, thiazolidinediones, an incretin receptor agonist (IRAs), glucagon like peptide 1 (GLP-1), glucagon like peptide 2 (GLP-2), and a peroxisome proliferator-activated receptor gamma (PPARγ) agonist. Such a treatment to a subject susceptible of developing Alzheimer's Disease may prevent the subject from developing (e.g., being diagnosed) with Alzheimer's Disease.

In some embodiments, the subject is determined to have Alzheimer's Disease where the one or more of the biomarkers detected in the biological sample are present in an amount at or above a threshold. In some embodiments, the threshold is the amount of the one or more biomarkers in a biological sample obtained from a subject without Alzheimer's Disease (or the average amount of the one or more biomarkers in a population of subjects without Alzheimer's Disease).

### BRIEF DESCRIPTION OF THE OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A-C show elevated levels of pIRS-636 detected in the CSF of individuals diagnosed with Alzheimer's Disease P-IRS1 (NB100-82003) (pSer636).

FIGS. 2A-C show elevated levels of pIRS-616 detected in the CSF of individuals diagnosed with Alzheimer's Disease P-IRS1 (701748) 26H8L6 (pSer616).

### DETAILED DESCRIPTION

The present disclosure provides methods for diagnosing or determining susceptibility to Alzheimer's Disease in a subject by obtaining a biological sample from the subject; detecting one or more biomarkers in the biological sample selected from the group consisting of: inflammation biomarkers, oxidative stress biomarkers, insulin resistance biomarkers, and autophagy biomarkers; and diagnosing the subject with Alzheimer's disease or being susceptible to Alzheimer's Disease where the one or more of the biomarkers are detected in the biological sample. Also provided herein are methods of treating a subject with Alzheimer's Disease, or susceptible to developing Alzheimer's Disease, comprising the step of administering to the subject a therapeutically effective amount of one or more agents for the treatment of inflammation, oxidative stress, insulin resistance, and/or autophagy.

### Definitions

Prior to setting forth the invention in detail, definitions of certain terms to be used herein are provided. Unless defined otherwise, all technical and scientific terms used herein have the same meaning as is commonly understood by one of skill in the art.

Where the term “comprising” is used in the present description and the claims, it does not exclude other elements or steps. For the purposes of the present invention, the term “consisting of” is considered to be a preferred embodiment of the term “comprising”.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In cases where numerical values are indicated in the context of the present disclosure, the skilled person will understand that the technical effect of the feature in question is ensured within an interval of accuracy, which typically encompasses a deviation of the numerical value given of ±10%, and preferably of ±5%. At the very least, and not as an attempt to limit the application of the doctrine of equivalents to the scope of the claims, each numerical parameter should at least be construed in light of the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventor for carrying out the disclosure. Of course, variations on these described embodiments will become apparent to those of ordinary skill in the art upon reading the foregoing description. The inventor expects skilled artisans to employ such variations as appropriate, and the inventor intends for the disclosure to be practiced other than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

Further definitions of terms will be given in the following in the context of which the terms are used. The following terms or definitions are provided solely to aid in the understanding of the invention. These definitions should not be construed to have a scope less than understood by a person of ordinary skill in the art.

As used herein, the term “subject” includes human and animals which are capable of suffering from or afflicted with dementia associated with a CNS disorder, including neurodegenerative diseases such as Alzheimer's Disease, or any disorder involving, directly or indirectly, Alzheimer's Disease. Examples of subjects include mammals, e.g., humans, non-human primates, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from Alzheimer's Disease or Alzheimer's Disease-associated dementia. In embodiments, the subject is a human between the ages of about 45-50, about 50 to about 55, about 55 to about 60, about 60 to about 65, about 65 to about 70, about 70 to about 75, about 75 to about 80, about 80 to about 85, about 85 to about 90, or greater than 90 years old.

As used herein, a biomarker “level in an individual without Alzheimer's disease” includes, for example, a biomarker level in an individual who is representative of an average level of the biomarker in a population of individuals without Alzheimer's disease. Thus, a level in an individual without Alzheimer's disease may be determined by, for example, determining an average level of the biomarker in a population of individuals without Alzheimer's disease.

As used herein, a biomarker “level in an individual that is not at risk of developing Alzheimer's disease” includes, for example, a biomarker level in an individual who is representative of an average level of the biomarker in a population of individuals that are not at risk of developing Alzheimer's disease. Thus, a level in an individual that is not at risk of developing Alzheimer's disease may be determined by, for example, determining an average level of the biomarker in a population of individuals that are not at risk of developing Alzheimer's disease.

As used herein, a “sample” is comprised of biologic material isolated from a subject and includes, without limitation, blood, serum, tissue, plasma or cerebrospinal fluid. Certain embodiments provide methods for collection of a sample (e.g., blood and other materials) useful for diagnostic purposes.

In some embodiments, a sample, or biological sample, according to the disclosure contains a population of cells or cell fragments, including without limitation cell membrane components, exosomes, and sub-cellular components. The cells may be a homogenous population of cells, such as isolated cells of a particular type, or a mixture of different cell types, such as from a biological fluid or tissue of a human or mammalian or other species subject. Still other samples for use in the methods and with the compositions include, without limitation, blood samples, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal or rectal). Still other samples include a blood-derived or biopsy-derived biological sample of tissue or a cell lysate (i.e., a mixture derived from tissue and/or cells). Other suitable tissue includes hair, fingernails and the like. Still other samples include libraries of antibodies, antibody fragments and antibody mimetics like affibodies. Such samples may further be diluted with saline, buffer or a physiologically acceptable diluent. Alternatively, such samples are concentrated by conventional means. Still other samples can be synthesized or engineered collections of chemical molecules, proteins, antibodies or any other of the targets described herein. A sample is often obtained from, or derived from a specific source, subject or patient.

In a preferred embodiment, the biological sample used for determining the level of one or more biomarkers is a sample containing circulating biomarkers, e.g., extracellular biomarkers. Extracellular biomarkers freely circulate in a wide range of biological material, including bodily fluids, such as fluids from the circulatory system, e.g., a blood sample or a lymph sample, or from another bodily fluid such as CSF, urine or saliva. Accordingly, in some embodiments, the biological sample used for determining the level of one or more biomarkers is a bodily fluid, for example, blood, fractions thereof, serum, plasma, urine, saliva, tears, sweat, semen, vaginal secretions, lymph, bronchial secretions, CSF, etc. In some embodiments, the sample is a sample that is obtained non-invasively. In some embodiments, the sample is obtained from a bodily fluid other than CSF.

### Diagnosis of Determining Susceptibility to Alzheimer's Disease

The present disclosure provides methods for diagnosing a neurodegenerative disease or disorder (e.g., Alzheimer's Disease) in a subject in need thereof or determining susceptibility of a subject in need thereof to developing the neurodegenerative disease or disorder (e. g, Alzheimer's Disease) by obtaining a biological sample from the subject; detecting one or more biomarkers in the biological sample selected from the group consisting of: inflammation biomarkers, oxidative stress biomarkers, insulin resistance biomarkers, and autophagy biomarkers; and diagnosing the subject with the neurodegenerative disease or disorder, or susceptible to developing the neurodegenerative disease or disorder, where one or more of the biomarkers are detected in the biological sample.

In some embodiments, the level of a biomarker in a sample of a subject is compared to the normal level of the biomarker in a subject, or subjects, without a neurodegenerative disease (e.g., Alzheimer's) or cancer, or to a reference standard. The normal level of a biomarker in a sample, or reference standard, can be an average level of a biomarker in samples of one or more healthy subjects (e.g., subjects with a CDR score of 0), such as subjects in the same age group and, optionally, of the same gender and/or ethnicity.

In other embodiments, a normal level of a biomarker in a sample can be an average level of a biomarker in samples of one or more healthy subjects having a Mini-Mental State Examination (“MMSE”) score between 27 and 30, such as subjects in the same age group and, optionally, of the same gender. During the MMSE, a physician or other medical professional asks a patient a series of questions that are designed to test a range of everyday mental skills. Questions commonly asked include, for example, remembering and repeating the names of three common objects, stating the year, date, season, and day of the week, counting backwards from 100 in increments of 7, spelling the word “world” backwards, naming familiar objects as the examiner points to them, identifying the location of the examiner's office, repeating a common phrase after it is stated by the Examiner, copying a picture of two interlocking shapes, and following a three-part series of instructions (e.g., pick up a piece of paper, fold it in half, and place it on the floor). The maximum score on the MMSE examination is 30 points. In general, a patient with an MMSE score of 27-30 is considered to have no cognitive impairment, a patient with an MMSE score of 21-26 is considered to have mild cognitive impairment, a patient with an MMSE score of 11-20 is considered to have moderate cognitive impairment, and a patient with an MMSE score of 0-10 is considered to have severe cognitive impairment. In certain embodiments, a patient with an MMSE score of 0-16 is considered to have advanced (moderately severe to severe) Alzheimer's disease.

In one embodiment, the difference in the level of a biomarker is an increase relative to a normal control sample. A suitable control may also be a reference standard. A reference standard serves as a reference level for comparison, such that a samples from a subject can be compared to the reference standard in order to infer the Alzheimer's Disease status of the subject. A reference standard may be representative of the level of one or more biomarkers in a known subject, e.g., a subject known to be a normal subject, or a subject known to have Alzheimer's Disease.

Likewise, a reference standard may be representative of the level of one or more biomarkers in a population of known subjects, e.g., a population of subjects known to be normal subjects, or a population of subjects known to have Alzheimer's Disease. The reference standard may be obtained, for example, by pooling samples from a plurality of individuals and determining the level of one or more biomarkers in the pooled samples, to thereby produce a standard over an averaged population. Such a reference standard represents an average level of a biomarker among a population of individuals.

A reference standard may also be obtained, for example, by averaging the level of a biomarker determined to be present in individual samples obtained from a plurality of individuals. Such a standard is also representative of an average level of a biomarker among a population of individuals.

A reference standard may also be a collection of values each representing the level of a biomarker in a known subject in a population of individuals. In certain embodiments, test samples may be compared against such a collection of values in order to infer the Alzheimer's Disease status of a subject.

In certain embodiments, the reference standard is an absolute value. In such embodiments, test samples may be compared against the absolute value in order to infer the Alzheimer's Disease status of a subject. In a one embodiment, a comparison between the level of one or more biomarkers in a sample relative to a suitable control is made by executing a software classification algorithm. The skilled person can readily envision additional suitable controls that may be appropriate depending on the assay in question. The aforementioned suitable controls are exemplary and are not intended to be limiting.

A subject having an increased level of the one or more biomarkers as compared to a normal subject may have Alzheimer's Disease, including early-stage Alzheimer's Disease, moderate or mid-stage Alzheimer's Disease, or severe or late-stage Alzheimer's Disease. In one embodiment, the level of one or more biomarkers may be used to diagnose Alzheimer's disease in a subject having symptoms characteristic of early-stage Alzheimer's Disease, also known as prodromal Alzheimer's Disease.

In another embodiment, the level of the one or more biomarkers may be used to diagnose Alzheimer's Disease in a subject having symptoms characteristic of “moderately severe cognitive decline,” also referred to as “moderate” or “mid-stage” Alzheimer's Disease. Moderately severe cognitive decline is characterized by major gaps in memory and the emergence of deficits in cognitive function. At this stage, some assistance with day-to-day activities is indicated.

In another embodiment, the level of the one or more biomarkers may be used to diagnose Alzheimer's Disease in a subject having symptoms characteristic of “severe cognitive decline,” also referred to as “moderate” or “mid-stage” Alzheimer's Disease. In severe cognitive decline, memory difficulties continue to worsen, significant personality changes may emerge, and affected individuals typically need extensive help with customary daily activities.

### Treatment of Alzheimer's Disease

The present disclosure provides methods for treating a subject determined to have Alzheimer's Disease or a subject determined to be susceptible to developing Alzheimer's Disease. Such methods may comprise administering to the subject a therapeutically effective amount of one or more agents for the treatment of inflammation, oxidative stress, insulin resistance, and/or autophagy.

As used herein, an Alzheimer's Disease treatment comprises prescribing or administering one or more therapeutic interventions to slow, prevent, reverse, or change disease progression. An Alzheimer's Disease treatment may be a drug or non-drug treatment. In some embodiments, an Alzheimer's Disease treatment, or therapeutic, may treat one or more symptoms of disease. For example, a therapeutic intervention may comprise administering a therapeutically effective amount of at least one Alzheimer's Disease therapeutic drug to the subject. In certain embodiments, the Alzheimer's Disease therapeutic may be Razadyne® (galantamine), Exelon® (rivastigmine), Aricept® (donepezil), Namenda® (memantine), or a pharmaceutically acceptable salt or ester thereof. In a particular embodiment, an Alzheimer's Disease treatment according to the disclosure comprises administering the therapeutic Aducanumab (Aduhelm™). In some embodiments, an Alzheimer's Disease treatment comprises treatment with Suvorexant (Belsomra®), Citalopram (Celexa®), Fluoxetine (Prozac®), Paroxeine (Paxil®), Sertraline (Zoloft®), Trazodone (Desyrel®), Lorazepam (Ativan®), Oxazepam (Serax®), Aripiprazole (Abilify®), Clozapine (Clozaril®), Haloperidol (Haldol®), Olanzapine (Zyprexa®), Quetiapine (Seroquel®), Risperidone (Risperdal®), Ziprasidone (Geodon®), or Carbamazepine (Tegretol®). In other embodiments, an Alzheimer's Disease treatment may comprise a non-drug therapeutic regimen, such as a behavioral therapy regimen.

The Alzheimer's Disease therapeutics may be administered to a subject using a pharmaceutical composition. Suitable pharmaceutical compositions comprise an Alzheimer's Disease therapeutic (or a pharmaceutically acceptable salt or ester thereof), and optionally comprise a pharmaceutically acceptable carrier, such as a pharmaceutical composition comprising galantamine, rivastigmine, donepezil or a pharmaceutically acceptable salt or ester of any of the foregoing (e.g., galantamine hydrobromide, rivastigmine tartrate, donepezil hydrochloride). In certain embodiments, these compositions optionally further comprise one or more additional therapeutic agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66:1-19 (1977), incorporated herein by reference. The salts can be prepared in situ during the final isolation and purification of the compounds of the invention, or separately by reacting a free base or free acid function with a suitable reagent. For example, a free base function can be reacted with a suitable acid. Furthermore, where the compounds carry an acidic moiety, suitable pharmaceutically acceptable salts thereof may, include metal salts such as alkali metal salts, e.g. sodium or potassium salts; and alkaline earth metal salts, e.g. calcium or magnesium salts.

The term “pharmaceutically acceptable ester”, as used herein, refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. As described above, the pharmaceutical compositions may additionally comprise a pharmaceutically acceptable carrier. The term carrier includes any and all solvents, diluents, or other liquid vehicle, dispersion or suspension aids, surface active agents, isotonic agents, thickening or emulsifying agents, preservatives, solid binders, lubricants and the like, suitable for preparing the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The terms “treat” or “treating” are used herein to mean to relieve, reduce or alleviate at least one symptom of a disease in a subject. For example, in relation to Alzheimer's Disease, the term “treat” includes relieving, reducing, or alleviating cognitive impairment (such as impairment of memory and/or orientation) or impairment of global functioning (overall functioning, including activities of daily living) and/or slowing down or reversing the progressive deterioration in global or cognitive impairment. Accordingly, the term “treat” also encompasses delaying or preventing onset prior to clinical manifestation of a disease or symptom of a disease and/or reducing the risk of developing or worsening of a symptom of a disease.

In some embodiments, administration of treatment for Alzheimer's is followed by monitoring of the level of a biomarker and, optionally, comparing the level of a biomarker to the normal level of a biomarker. In some embodiments, administration of a first dose of a treatment for Alzheimer's is followed by determining the level of a biomarker, and if the level of a biomarker is increased over the normal level, administering a second treatment for Alzheimer's at a higher dose (e.g., 1.25, 1.5, 1.75, 2, 2.5, 3, 4, 5, 6, 7, 8, 9, or 10 times higher) than the first dose.

### Detection Methods

In certain embodiments, the levels of the one or plurality of the inflammation biomarkers, oxidative stress biomarkers, insulin resistance biomarkers, and/or autophagy biomarkers in the sample are detected using any method known in the art. In an embodiment, the biomarkers are detected using mass spectrometry. In particular embodiments, the levels of biomarkers are detected using selected reaction monitoring mass spectrometry (SRM-MS). In other embodiments, the levels of the one or plurality of the biomarkers in the sample are detected using other quantitative mass spectrometry techniques, including, without limitation, spectral counting, isobaric mass tagging, or ion mobility mass spectrometry.

In further embodiments, the absolute concentration of the one or a plurality of biomarkers is determined. In some embodiments, absolute concentration of the one or a plurality of biomarkers is determined using SRM-MS in combination with the AQUA method.

In still other embodiments, the levels of the one or plurality of the biomarkers is determined by electrophoresis. For example, in some embodiments, the level of the biomarker is determined by, without limitation, one-or two-dimensional electrophoresis, or capillary electrophoresis. Those skilled in the art will recognize still further quantitative electrophoresis methods suitable for practicing the present disclosure.

The level of a biomarker in a sample can be determined by immunologic or affinity-based methods. For example, the level of a biomarker in a sample can be determined by assessing (e.g., quantifying) the level of the biomarker in the sample using, e.g., immunohistochemical analysis, Western blotting, ELISA, immunoprecipitation, flow cytometry analysis, or any other technique known in the art or described herein. In particular embodiments, the level of a biomarker is determined by a method capable of quantifying the amount of a biomarker present in a sample of a patient, and/or capable of detecting the correction of the level of a biomarker following treatment.

In some embodiments, a biomarker of the disclosure is contacted with a ligand that has affinity for the biomarker. The “ligand” used in these compositions and methods refers to any naturally occurring or synthetic biological or chemical molecule which is used to bind specifically to a biomarker. The binding can be covalently or non-covalent, i.e., conjugated or by any known means taking into account the nature of the ligand and its respective target. A ligand may independently be selected from a peptide, a protein, an antibody or antibody fragment (e.g., an antigen binding portion of an antibody), an antibody mimetic, an affibody, a ribo- or deoxyribonucleic acid sequence, an aptamer, a lipid, a polysaccharide, a lectin, or a chimeric molecule formed of multiples of the same or different ligands. Additional non-limiting examples of a ligand include a Fab, Fab′, F(ab′)2, Fv fragment, single-chain Fv (scFv), diabody (Dab), synbody, nanobodies, BiTEs, SMIPs, DARPins, DNLs, Duocalins, adnectins, fynomers, Kunitz Domains Albu-dabs, DARTs, DVD-IG, Covx-bodies, peptibodies, scFv-Igs, SVD-Igs, dAb-Igs, Knob-in-Holes, or combinations thereof. In some embodiments, a ligand is a recombinant or naturally occurring protein. In certain embodiments, a ligand is a monoclonal or polyclonal antibody, or fragment thereof. In some embodiments, the ligand(s) of the constructs can also be directly labeled with one or more detectable labels, such as fluorophores (see labels discussed below) that can be measured by methods independent of the methods of measuring or detecting the polymer construct described otherwise herein.

The term “antibody” as used herein is intended to include fragments thereof which are also specifically reactive with a subject polypeptide. Antibodies can be fragmented using conventional techniques and the fragments screened for utility in the same manner as described above for whole antibodies. For example, F(ab) 2 fragments can be generated by treating antibody with pepsin. The resulting F(ab) 2 fragment can be treated to reduce disulfide bridges to produce Fab fragments. An antibody is further intended to include bispecific, single-chain, chimeric, humanized and fully human molecules having affinity for a biomarker of the disclosure conferred by at least one CDR region of the antibody. An antibody may further comprise a label attached thereto and able to be detected (e.g., the label can be a radioisotope, fluorescent compound, enzyme or enzyme co-factor).

In certain embodiments, the antibody is a recombinant antibody, which term encompasses any antibody generated in part by techniques of molecular biology, including CDR grafted or chimeric antibodies, human or other antibodies assembled from library-selected antibody domains, single chain antibodies and single domain antibodies (e.g., human VH proteins or camelid VHH proteins). In certain embodiments, an antibody can be a monoclonal antibody, and in certain embodiments. For example, a method for generating a monoclonal antibody that binds specifically to a biomarker of the disclosure may comprise administering to a mouse an amount of an immunogenic composition comprising the antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the antigen. Once obtained, a hybridoma can be propagated in a cell culture, optionally in culture conditions where the hybridoma-derived cells produce the monoclonal antibody that binds specifically to the antigen. The monoclonal antibody may be purified from the cell culture.

In some embodiments, phospho-specific antibodies provided by the disclosure are specifically reactive with a phosphorylated form of a biomarker. As used herein, “specifically reactive with” in reference to an antibody is intended to mean, as is generally understood in the art, that the antibody is selective between the antigen of interest (e.g., a phosphorylated form of a biomarker) and other antigens that are not of interest (e.g. the unphosphorylated biomarker, or the total amount of the biomarker present in the sample). Monoclonal antibodies generally have a greater tendency (as compared to polyclonal antibodies) to discriminate effectively between the desired antigens and cross-reacting polypeptides. One characteristic that influences the specificity of an antibody: antigen interaction is the affinity of the antibody for the antigen. Although the desired specificity may be reached with a range of different affinities, generally preferred antibodies will have an affinity (a dissociation constant) of about 1×10⁻⁸ M or less.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore™ binding assay, Biacore AB, Uppsala, Sweden), sandwich assays (e.g., the paramagnetic bead system of IGEN International, Inc., Gaithersburg, Md.), Western blots, immunoprecipitation assays, and immunohistochemistry.

In one embodiment, the level of a biomarker in a tissue sample is determined by assessing (e.g., quantifying) protein expression of a biomarker in the sample using ELISA. For example, a biomarker can be identified and quantified in the human serum using sandwich ELISA method. The sandwich ELISA method for use in determining the level of a biomarker in a tissue sample can comprise coating of ELISA plates with one or more anti-biomarker antibodies, contacting the plates with the sample (e.g., human serum), and detecting biomarker ligand in the sample.

Phospho-specific antibodies for use in assays that measure the levels of a biomarker in a sample are known in the art or could be readily developed using approaches known to those of skill in the art.

In some embodiments, a ligand or antibody of the disclosure further comprises a detectable label, meaning a reagent, moiety or compound capable of providing a detectable signal, depending upon the assay format employed. A label may be associated with a ligand or antibody only, or with ligand or antibody in complex with one or more features of the disclosure. Such labels are capable, alone or in combination with other compositions or compounds, of providing a detectable signal. In one embodiment, the labels are desirably interactive to produce a detectable signal. In certain embodiments, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength. Still other label systems that may be utilized in the described methods and constructs are detectable by other means, e.g., colored latex microparticles (Bangs Laboratories, Indiana) in which a dye is embedded may be used in place of enzymes to provide a visual signal indicative of the presence of the labeled ligand or antibody in applicable assays. Still other labels include fluorescent compounds, fluorophores, radioactive compounds or elements. In one embodiment, a fluorescent detectable fluorochrome, e.g., fluorescein isothiocyanate (FITC), phycoerythrin (PE), allophycocyanin (APC), coriphosphine-O (CPO) or tandem dyes, PE-cyanin-5 or -7 (PC5 or PC7)), PE-Texas Red (ECD), PE-cyanin-5.5, rhodamine, PerCP, and Alexa dyes. Combinations of such labels, such as Texas Red and rhodamine, FITC +PE, FITC +PECy5 and PE +PECy7, among others may be used depending upon assay method. The selection and/or generation of suitable labels for use in labeling the ligand and/or any component of a ligand or antibody of the disclosure is within the skill of the art, provided with this specification.

### 1. Quantitative Immuno PCR

In embodiments, the biomarkers of the disclosure are detected by a quantitative immuno-PCR (qIPCR) assay. Accordingly, in certain embodiments the biomarkers of the disclosure are detected by a ligand, e.g. an antibody, that is attached to an oligonucleotide bearing a primer site and a barcode unique to the ligand.

By the term “attachment” or “attach” as used herein to describe the interaction between the components of the constructs is meant covalent attachments or a variety of non-covalent types of attachment. Other attachment chemistries useful in assembling the constructs described herein include, but are not limited to, thiol-maleimide, thiol-haloacetate, amine-NHS, amine-isothiocyanate, azide-alkyne (CuAAC), tetrazole-cyclooctene (iEDDA) (See, e.g., reference 24 and other references therein). In one embodiment, each polymer construct is linked to the ligand by an irreversible covalent link. In another embodiment, each polymer construct is linked to the ligand by a cleavable covalent link, for example a disulfide link or a photocleavable linker.

Thus, in some embodiments a linker molecule with bispecific binding affinity for nucleic acids and antibodies is used to attach a DNA, RNA, DNA/RNA hybrid, or their fragment, analogue or derivative molecule used as a marker, specifically to an antigen-antibody complex, resulting in the formation of a antigen-antibody-linker-DNA conjugate. A segment of the attached marker is amplified enzymatically (such as by a polymerase chain reaction with appropriate primers). The presence of specific products of polymerase chain reaction or other amplification methods demonstrates that marker molecules are attached specifically to antigen-antibody complexes and in turn, this indicates the presence of antigen.

The ligand can be attached to the construct oligonucleotide sequence at its 5′ end or at any other portion, provided that the attachment or conjugation does not prevent the functions of the components of the construct oligonucleotide sequence.

In general, the oligonucleotide can be any length that accommodates the lengths of its functional components. In one embodiment, the oligonucleotide is between 20 and 100 monomeric components, e.g., nucleic acid bases, in length. In some embodiments, the oligonucleotide is at least 20, 30, 40, 50, 60, 70, 80, 90 or over 100 monomeric components, e.g., nucleic acid bases, in length. In other embodiments, the oligonucleotide is 200 to about 400 monomeric components, e.g., nucleotides, in length.

In certain embodiments, the polymer construct is generally made up of deoxyribonucleic acids (DNA). In some embodiments, the oligonucleotide is a DNA sequence. In other embodiments, the oligonucleotide, or portions thereof, comprises modified DNA bases. Modification of DNA bases are known in the art, and can include chemically modified bases including labels.

In other embodiments, the oligonucleotide, or portions thereof, comprises ribonucleic acid (RNA) sequences or modified ribonucleotide bases. Modification of RNA bases are known in the art, and can include chemically modified bases including labels. In still other embodiments, different portions of the oligonucleotide sequence can comprise DNA and RNA, modified bases, or modified polymer connections (including but not limited to PNAs and LNAs).

As used herein, the term “primer sequence” refers to a functional component of the construct oligonucleotide sequence which itself is an oligonucleotide or polynucleotide sequence that provides an annealing site for amplification of the oligonucleotide sequence. The primer sequence can be formed of polymers of DNA, RNA, PNA, modified bases or combinations of these bases, or polyamides, etc. In one embodiment, the primer sequence is about 10 of such monomeric components, e.g., nucleotide bases, in length. In other embodiments, the primer sequence is at least about 5 to 100 monomeric components, e.g., nucleotides, in length. Thus in various embodiments, the primer sequence is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In certain embodiments, e.g., in multiplexed biomarker detection according to the disclosure, multiple oligonucleotide sequences are attached to multiple ligands, e.g. antibodies, and the primer sequence can be the same or different, depending upon the techniques intended to be used for amplification. In certain embodiments, the primer sequence can be a generic sequence suitable as a annealing site for a variety of amplification technologies.

Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependant amplification (HDA), ramification amplification method and RNA-seq23.

As used herein, the term “barcode” describes a defined polymer, e.g., a polynucleotide, which when it is a functional element of the oligonucleotide, is specific for a single ligand. As used in the various methods described herein the term barcode can be a “cell barcode” or “substrate barcode”, which describes a defined polynucleotide, specific for identifying a particular cell or substrate, e.g., drop-seq microbead. In either embodiment, the barcode can be formed of a defined sequence of DNA, RNA, modified bases or combinations of these bases, as well as any other polymer defined above. In one embodiment, the barcode is about 2 to 4 monomeric components, e.g., nucleotide bases, in length. In other embodiments, the barcode is at least about 1 to 100 monomeric components, e.g., nucleotides, in length. Thus in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

In some embodiments, an oligonucleotide of the disclosure further comprises an anchor sequence designed to hybridize to another oligonucleotide sequence, e.g., a capture polymer, a capture oligonucleotide, a primer and the like. In certain embodiments, an anchor is designed for the purpose of generating a double-stranded construct oligonucleotide sequence. In some embodiments, the anchor is positioned at the 3′ end of an oligonucleotide sequence (e.g., a construct oligonucleotide sequence). In other embodiments, an anchor is positioned at the 5′ end of a construct oligonucleotide sequence. In some embodiments, each anchor is specific for its intended complementary sequence. For example, in certain embodiments, an anchor is configured to hybridize to a 3′ end of a capture oligonucleotide such that the 3′ end of the capture oligonucleotide acts as a primer that can generate a second complementary strand of the oligonucleotide in the presence of a polymerase. In certain embodiments, when the compositions or methods comprise multiple “first constructs”, each first construct has the same anchor sequence. In some embodiments, each additional anchor has a different additional sequence which hybridizes to a different complementary sequence. In other embodiments, each additional anchor may have the same anchor sequence as the first or other constructs, depending upon the assay method steps. When used in the various methods described herein, an anchor may hybridize to a free complementary sequence or with a complementary sequence that is immobilized on a substrate. In certain embodiments, the anchor can be formed of a sequence of monomers of the selected polymer, e.g., DNA, RNA, modified bases or combinations of these bases, PNAs, polyamides, etc. In one embodiment, an anchor is about 3 to 15 monomeric components, e.g., nucleotides, in length. In other embodiments, each anchor can be at least about 3 to 100 monomeric components, e.g., nucleotides, in length. In some embodiments, an anchor comprises 3 to 100, 3 to 50, 3 to 30, 5 to 30, 10 to 20, 5 to 20, or 5 to 15 monomeric components (e.g., nucleotides in length). In various embodiments, an Anchor is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

In some embodiments, an anchor sequence comprises or consists of a poly A sequence. In certain embodiments, a polyA sequence comprises a nucleic acid sequence comprising ten or more (e.g., 10-40, 10-30 or 10-20) consecutive adenosine nucleotides, derivatives or variants of an adenosine nucleotide, the like, or a combination thereof. In other embodiments, an anchor sequence comprises or consists of a poly T sequence. In another embodiment, an anchor sequence is a polyG sequence. In still other embodiments, an anchor sequence may be a random sequence provided that it can hybridize to its intended complementary sequence (e.g., a capture oligonucleotide, amplification primer, or the like). For example, in some embodiments a method described herein may utilize a plurality of oligonucleotides (e.g., a plurality of constructs comprising a ligand attached to an oligonucleotide), where some or all of the oligonucleotides comprise a different anchor (i.e., an anchor having a different nucleic acid sequence, or an anchor having a substantially different nucleic acid sequence). In some embodiments a method described herein may utilize a plurality of oligonucleotides (e.g., a plurality of constructs comprising a ligand attached to an oligonucleotide), where some or all of the oligonucleotides comprise the same anchor. In some embodiments a method described herein may utilize a plurality of oligonucleotides (e.g., a plurality of constructs comprising a ligand attached to an oligonucleotide), where some or all of the oligonucleotides comprise an anchor that is substantially identical (e.g., comprising a nucleic acid sequence that is substantially identical). In some embodiments a method described herein may utilize a plurality of oligonucleotides (e.g., a plurality of constructs comprising a ligand attached to an oligonucleotide), where some or all of the oligonucleotides comprise an anchor comprising a polyA sequence. In some embodiments, the polyA sequence of a plurality of anchors is substantially identical. As understood by one of skill in the art, polyA sequences that are substantially identical may differ substantially in length. In some embodiments, a polyA sequence (e.g, a polyA sequence of an anchor) is a nucleic acid configured to hybridize to a polyT sequence (e.g., an oligonucleotide or capture oligonucleotide comprising a polyT sequence). As understood by one of skill in the art, depending on hybridization conditions a polyA sequence may comprise one, two, three or four non-polyA nucleotides and still hybridize efficiently to a polyT sequence, thereby providing an annealed polyA-polyT complex comprising one, two, three or more mismatches. Accordingly, in some embodiments, a polyA sequence is a nucleic acid sequence comprising at least 70%, at least 75%, at least 80%, at least 85%, at least 90%, at least 95% or 100% adenosine nucleotides, adenosine analogs, adenosine variants or a combination thereof.

In some embodiments, an oligonucleotide comprises a polyT sequence. In some embodiments, a capture oligonucleotide comprises a polyT sequence (e.g., a 3′ polyT sequence). In some embodiments a method described herein may utilize a plurality of oligonucleotides (e.g., a plurality of capture oligonucleotides), where some or all of the oligonucleotides comprise a polyT sequence. In some embodiments, a polyT sequence of a plurality of oligonucleotides is substantially identical. In some embodiments, a plurality of capture oligonucleotides (e.g., a plurality of different capture oligonucleotides, e.g., different bead-specific capture oligonucleotides) comprise a polyT sequence that is substantially identical. As understood by one of skill in the art, polyT sequences that are substantially identical may differ substantially in length. In some embodiments, a polyT sequence comprises 3 to 100, 3 to 50, 3 to 30, 5 to 30, 10 to 20, 5 to 20, or 5 to 15 consecutive nucleotides (e.g., nucleotides in length). In certain embodiments, a polyT sequence comprises a nucleic acid sequence comprising three or more, ten or more, 3 to 100, 3 to 50, 3 to 30, 5 to 30, 10 to 20, 5 to 20, or 5 to 15 consecutive thymidine nucleotides, derivatives or variants of a thymidine nucleotide, the like, or a combination thereof. In some embodiments, a polyT sequence (e. g, a polyT sequence of a capture oligonucleotide) is a nucleic acid configured to hybridize to a polyA sequence. As understood by one of skill in the art, depending on hybridization conditions, a polyT sequence may comprise one, two, three or four non-thymidine nucleotides and still hybridize efficiently to a polyA sequence, thereby providing an annealed poly A-polyT complex comprising one, two, three or more mismatches. Accordingly, in some embodiments, a polyT sequence is a nucleic acid sequence comprising at least 70%, at least 75%, at least 80%, at least 85%, at least 90%, at least 95% or 100% thymidine nucleotides, thymidine analogs, thymidine variants or a combination thereof. In some embodiments, a polyT sequence comprises one or more uracil nucleotides, or derivative thereof.

In embodiments, the “linker” comprises any moiety used to attach or associate the ligand to the oligonucleotide sequence. Thus in one embodiment, the linker is a covalent bond. In another embodiment, the linker is a non-covalent bond. In another embodiment the linker is composed of at least one to about 25 atoms. Thus in various embodiments, the linker is formed of at least 2, 3, 4, 5, 6, 7, 8, 9, 10, 11, 12, 13, 14, 15, 16, 17, 18, 19, 20, 21, 22, 23, 24 or 25 atoms. In still another embodiment, the linker is at least one to about 60 nucleic acids. Thus in various embodiments, the linker is formed of a sequence of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, up to 60 nucleic acids. In yet another embodiment, the linker refers to at least one to about 30 amino acids. Thus, in various embodiments, the linker is formed of a sequence of at least 2, 3, 4, 5, 6, 7, 8, 9, 10, 11, 12, 13, 14, 15, 16, 17, 18, 19, 20, 21, 22, 23, 24, 25, 26, 27, 28, 29, up to about 30 amino acids.

In still other embodiments, the linker can be a larger compound or two or more compounds that associate covalently or non-covalently. In still other embodiments, the linker can be a combination of the linkers defined herein.

In some embodiments, the linkers used in the disclosure are cleavable. In other embodiments, the linkers used in the disclosure are non-cleavable. In certain embodiments, the linker is a cleavable linker, e.g., disulfide bond or photocleavable bond.

In certain exemplary embodiments, the linker comprises a complex of biotin bound to the construct oligonucleotide sequence by a disulfide bond, with streptavidin bound to the ligand, e.g., the antibody. In another embodiment, the biotin is bound to the ligand and the streptavidin is bound to the construct oligonucleotide sequence. The linker may be covalently attached or conjugated other than covalently to any oligonucleotide sequence portion of the construct. In yet another embodiment, when the ligand is a recombinant or synthesized antibody, the linker can be engineered into the antibody sequence to facilitate 1:1 coupling to the polymer construct, thereby simplifying manufacturing of the ligand, the construct and/or the polymer construct. For example, a Halotag® linker can be engineered into the selected ligand (e.g., antibody) or into the polymer construct or component, for such purposes. Additionally or alternatively, the ligand is linked to the polymer construct upon production in the same cell. See, e.g., the Halotag® protocols described by Flexi® Vector Systems Technical Manual (TM254—revised 5/17), copyright 2017 by Promega Corporation; and Janssen D. B., “Evolving haloalkaline dehalogenase”, Curr. Opin. Chem. Biol., 2004, 8:150-159.

In some embodiments, a streptavidin-protein A chimera is used as the linker molecule. The chimera has two independent specific binding abilities. One is its binding to biotin, derived from the streptavidin moiety, and the other is its binding to the Fc portion of an immunoglobulin G (IgG) molecule, derived from the protein A moiety. This bifunctional specificity both for biotin and antibody allows the specific conjugation of any biotinylated nucleic acid molecule to antigen-antibody complexes. Other linker molecules, such as any protein, peptide, nucleic acid marker chemically cross-linked to antibodies, or biotinylated marker nucleic acid cross-linked to biotinylated antibodies by streptavidin or avidin may be also advantageously utilized.

### 2. Digital Droplet PCR

In some embodiments, a ligand-oligonucleotide-biomarker complex isolated according to methods disclosed herein is amplified by digital droplet PCR. Thus, in embodiments, a population of oligonucleotides according to the disclosure may be encapsulated into droplets. In some embodiments, the oligonucleotides are encapsulated at relatively low concentrations, e.g., such that the droplets, on the average, contain less than one oligonucleotide per droplet. Accordingly, most or all of the oligonucleotides are amplified, e.g., substantially evenly.

In some embodiments, a plurality of primers may be added to the droplets to cause amplification, e.g., using droplet-based PCR or other techniques known to those of ordinary skill in the art. In some cases, there may be at least 3, at least 5, at least 10, at least 30, at least 50, at least 100, at least 300, at least 500, at least 1,000, at least 2,000, at least 3,000, at least 5,000, or at least 10,000, or more distinguishable primers present. This may be useful, for example, to ensure a large number of potential target oligonucleotides are amplified.

In some embodiments, the oligonucleotides in the amplified droplets may be determined or sequenced, e.g., using any of a variety of techniques. For instance, in one set of embodiments, the droplets may be broken and their contents pooled together, e.g., to create a pool of amplified oligonucleotides. The pool of amplified oligonucleotides may then be sequenced for quantitative determination, for example, using techniques such as Illumina sequencing, single-molecule real-time sequencing (e.g., Pacbio sequencing), nanopore sequencing, and their levels determined.

### Kits

In another aspect, the present invention provides kits for diagnosing Alzheimer's Disease status in a subject, which kits are useful for determining the level of one or more of biomarkers of inflammation, biomarkers of oxidative stress, biomarkers of insulin resistance, and/or biomarkers of autophagy.

Kits may include materials and reagents adapted to selectively detect the presence of a biomarker diagnostic for Alzheimer's Disease in a sample derived from a subject. For example, in one embodiment, the kit may include one or more phospho-specific ligands, such as one or more phospho-specific antibodies, capable of diagnosing Alzheimer's Disease in a sample derived from a subject. The kit may include reagents useful for performing an assay to detect one or more biomarkers, for example, reagents which may be used to detect a phospho-specific ligand bound to a biomarker of the disclosure.

In a further embodiment, the kit may contain instructions for suitable operational parameters in the form of a label or product insert. For example, the instructions may include information or directions regarding how to collect a sample, how to determine the level of one or more biomarkers in a sample, or how to correlate the level of one or more biomarkers in a sample with the Alzheimer's Disease status of a subject.

In another embodiment, the kit can contain one or more containers with miRNA biomarker samples, to be used as reference standards, suitable controls, or for calibration of an assay to detect the miRNA biomarkers in a test sample.

Computer systems are also provided having one or more processors and memory storing one or more programs for execution by the one or more processors. Such a system includes memory-storing instructions for causing the computer system to perform any of the methods described herein including a portion of any of the methods disclosed herein.

Features of the embodiments described herein can be implemented in, using, or with the assistance of a computer program product, such as a storage medium (media) or computer readable storage medium (media) having instructions stored thereon/in which can be used to program a processing system to perform any of the features presented herein. The storage medium (e.g., the memory) can include, but is not limited to, high-speed random-access memory, such as DRAM, SRAM, DDR RAM or other random access solid state memory devices, and may include non-volatile memory, such as one or more magnetic disk storage devices, optical disk storage devices, flash memory devices, or other non-volatile solid state storage devices. In some embodiments, the memory may include one or more storage devices remotely located from the CPU(s). The memory, or alternatively the non-volatile memory device(s) within these memories, comprises a non-transitory computer readable storage medium.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1: A method of treating a subject with Alzheimer's disease, the method comprising: administering to the subject an effective amount of one or more agents for the treatment of inflammation, oxidative stress, insulin resistance, and/or autophagy.

Clause 2: The method of Clause 1, wherein the subject is administered one or more agents for the treatment of inflammation.

Clause 3: The method of Clause 2, wherein the agent is rapamycin, minocycline, thalidomide or its analogues, rosiglitazone, an NSAID, or pioglitozone.

Clause 4: The method of Clause 3, wherein the NSAID is naproxen or rofecoxib.

Clause 5: The method of Clause 1, wherein the subject is administered one or more agents for the treatment of oxidative stress.

Clause 6: The method of Clause 5, wherein the agent is hydrogen peroxide.

Clause 7: The method of Clause 1, wherein the subject is administered one or more agents for the treatment of insulin resistance.

Clause 8: The method of Clause 7, wherein the agent is insulin, thiazolidinediones, an incretin receptor agonist (IRAs), glucagon like peptide 1 (GLP-1), glucagon like peptide 2 (GLP-2), or a peroxisome proliferator-activated receptor gamma (PPARγ) agonist.

Clause 9: The method of Clause 8, wherein the IRA is liraglutide.

Clause 10: The method of Clause 8, wherein the PPARγ) agonist is rosiglitazone.

Clause 11: The method of Clause 1, wherein the subject is administered one or more agents for the treatment of autophagy.

Clause 12: The method of Clause 11, wherein the agent inhibits Pik3.

Clause 13: The method of Clause 11, wherein the agent is rapamycin, everolimus, temsirolimus, metformin, trehalose, curcumin or analogues thereof, aspirin, gemfibrozil, Wy14643, cinnamic acid, gypenoside XVII, ouabain, fisetin, or flubendazole.

Clause 14: A method of treating a subject with or suspected of having Alzheimer's disease, the method comprising: obtaining a biological sample from the subject with or suspected of having Alzheimer's disease; detecting one or more biomarkers in the biological sample selected from the group consisting of: inflammation biomarkers, oxidative stress biomarkers, insulin resistance biomarkers, and autophagy biomarkers; and administering to the subject an effective amount of one or more agents that treat inflammation where one or more inflammation biomarkers are detected in the biological sample, one or more agents that treat oxidative stress where one or more oxidative stress biomarkers are detected in the biological sample, one or more agents that treat insulin resistance where one or more insulin resistance biomarkers are detected in the biological sample, and/or one or more agents that treat autophagy where one or more autophagy biomarkers are detected in the biological sample.

Clause 15: The method of Clause 14, wherein the biological sample is cerebral spinal fluid (CSF).

Clause 16: The method of Clause 14, wherein the biological sample is blood.

Clause 17: The method of Clause 14, wherein the inflammation biomarker is p38 MAPK, C-reactive protein (CRP), IL6, or TNFα.

Clause 18: The method of Clause 14, wherein the insulin resistance biomarker is insulin receptor substrate 1 (IRS-1).

Clause 19: The method of Clause 14, wherein the IRS-1 is phosphorylated at Ser616 Ser636, and/or Ser312.

Clause 20: The method of Clause 14, wherein the autophagy biomarker is p62.

Clause 21: The method of Clause 14, wherein the oxidative stress biomarker for is DNA damage, cleaved caspase 3, gamma-H2AX, BAD, or BAX.

Clause 22: The method of Clause 14, wherein the subject is determined to have Alzheimer's disease where the one or more of the biomarkers detected in the biological sample are present in an amount at or above a threshold.

Clause 23: The method of Clause 22, wherein the threshold is the amount of the one or more biomarkers in a biological sample obtained from a subject without Alzheimer's disease.

Clause 24: The method of Clause 14, wherein the subject is administered one or more agents for the treatment of inflammation.

Clause 25: The method of Clause 24, wherein the agent is rapamycin, minocycline, thalidomide or its analogues, rosiglitazone, an NSAID, or pioglitozone.

Clause 26: The method of Clause 25, wherein the NSAID is naproxen or rofecoxib.

Clause 27: The method of Clause 14, wherein the subject is administered one or more agents for the treatment of oxidative stress.

Clause 28: The method of Clause 27, wherein the agent is hydrogen peroxide.

Clause 29: The method of Clause 14, wherein the subject is administered one or more agents for the treatment of insulin resistance.

Clause 30: The method of Clause 29, wherein the agent is insulin, thiazolidinediones, an incretin receptor agonist (IRAs), glucagon like peptide 1 (GLP-1), glucagon like peptide 2 (GLP-2), or a peroxisome proliferator-activated receptor gamma (PPARγ) agonist.

Clause 31: The method of Clause 30, wherein the IRA is liraglutide.

Clause 32: The method of Clause 30, wherein the PPARγ) agonist is rosiglitazone.

Clause 33: The method of Clause 14, wherein the subject is administered one or more agents for the treatment of autophagy.

Clause 34: The method of Clause 33, wherein the agent inhibits Pik3.

Clause 35: The method of Clause 33, wherein the agent is rapamycin, everolimus, temsirolimus, metformin, trehalose, curcumin or analogues thereof, aspirin, gemfibrozil, Wy14643, cinnamic acid, gypenoside XVII, ouabain, fisetin, or flubendazole.

Clause 36: A method for diagnosing or determining susceptibility to Alzheimer's Disease a subject with Alzheimer's disease, the method comprising: obtaining a biological sample from the subject; detecting one or more biomarkers in the biological sample selected from the group consisting of: inflammation biomarkers, oxidative stress biomarkers, insulin resistance biomarkers, and autophagy biomarkers; and diagnosing the subject with Alzheimer's disease where one or more of the biomarkers is detected in the biological sample.

Clause 37: The method of Clause 36 wherein the biological sample is cerebral spinal fluid (CSF).

Clause 38: The method of Clause 36, wherein the biological sample is blood.

Clause 39: The method of Clause 36, wherein the inflammation biomarker is p38 MAPK.

Clause 40: The method of Clause 36, wherein the insulin resistance biomarker is insulin receptor substrate 1 (IRS-1).

Clause 41: The method of Clause 40, wherein the IRS-1 is phosphorylated at Ser616.

Clause 42: The method of Clause 40, wherein the IRS-1 is phosphorylated at Ser636.

Clause 43: The method of Clause 40, wherein the IRS-1 is phosphorylated at Ser616 and Ser636.

Clause 44: The method of Clause 36, wherein the autophagy biomarker is p62.

Clause 45: The method of Clause 36, wherein the oxidative stress biomarker for is DNA damage, cleaved caspase 3, gamma-H2AX, BAD, or BAX.

Clause 46: The method of Clause 36, wherein the subject is determined to have Alzheimer's disease where the one or more of the biomarkers detected in the biological sample are present in an amount at or above a threshold.

Clause 47: The method of Clause 46, wherein the threshold is the amount of the one or more biomarkers in a biological sample obtained from a subject without Alzheimer's disease.

This disclosure is further illustrated by the following examples which are provided to facilitate the practice of the disclosed methods. These examples do not limit the scope of the disclosure in any way.

### EXAMPLES

### Example 1: Detection of pIRS-636 in the CSF of Individuals Diagnosed With Alzheimer's Disease

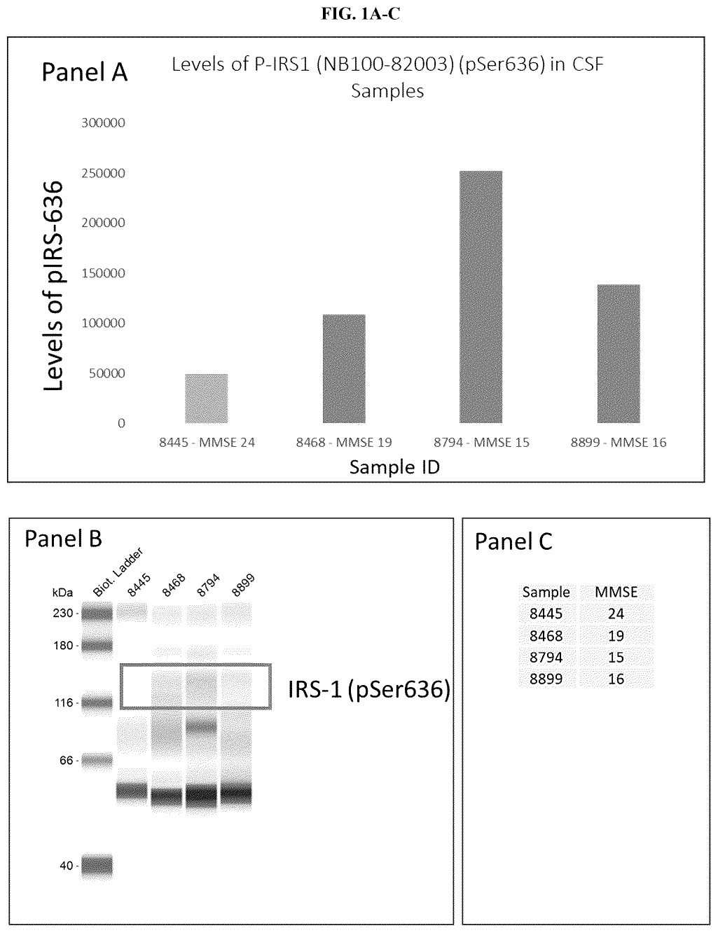

The CSF of human patients diagnosed with Alzheimer's Disease (AD) was collected. Levels of pIRS-636 were measured by western blots using an antibody specific for pIRS-636 (Thermo Fisher monoclonal antibody clone NB100-82003; Catalog #701748). Levels of pIRS-616 are defined using the Area Under the Curve of each individual western blot lane. AD samples are shown in the blue bars and a normal sample shown in the grey bar (see, FIG. 1 (Panel A)). The image of the Western blot data is provided in FIG. 1 (Panel B). The corresponding pIRS molecular weight is 131 kDa for P-IRS1 (NB100-82003) (pSer636). The sample name and corresponding Mini-Mental State Examination (MMSE) Score are noted in FIG. 1 (Panel C). Western blot analysis shows that the levels of phosphorylated IRS1 (Serine 636; pIRS-636) are elevated in the CSF of individuals diagnosed with Alzheimer's Disease.

### Example 2: Detection of pIRS-616 in the CSF of Individuals Diagnosed with Alzheimer's Disease

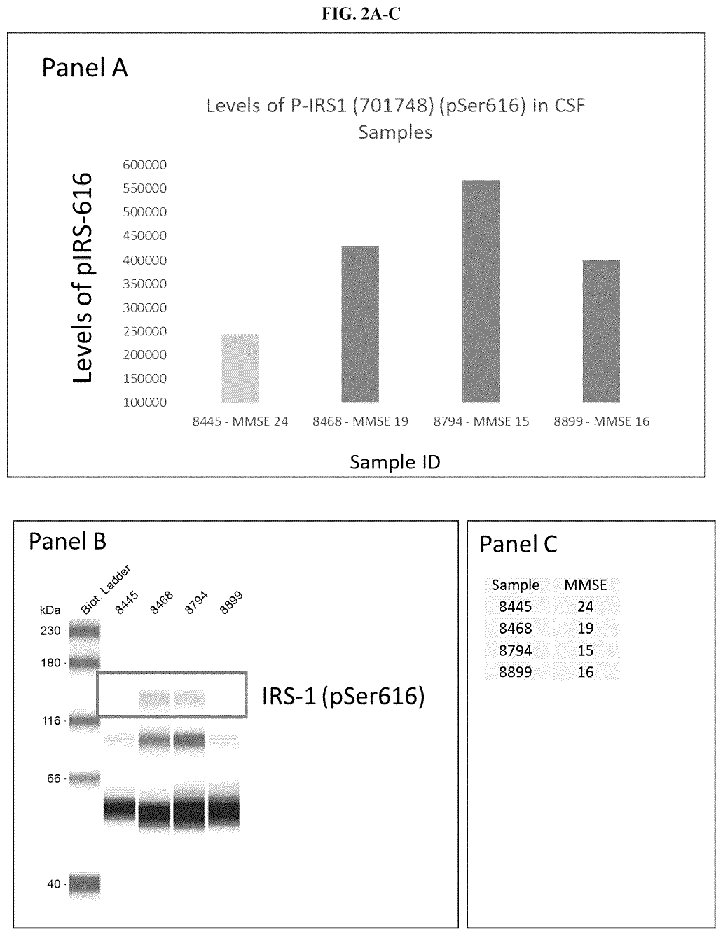

The CSF of human patients diagnosed with Alzheimer's Disease (AD) was collected. Levels of pIRS-616 were measured by western blots using an antibody specific for pIRS-616 (Thermo Fisher monoclonal antibody clone 26H8L6; Catalog #701748). Levels of pIRS-616 are defined using the Area Under the Curve of each individual western blot lane. AD samples are shown in the blue bars and a normal sample shown in the grey bar (see, FIG. 2 (Panel A)). The image of the Western blot data is provided in FIG. 2 (Panel B). The corresponding pIRS molecular weight is 131 kDa for P-IRS1 (701748) (pSer616). The sample name and corresponding Mini-Mental State Examination (MMSE) Score are noted in FIG. 2 (Panel C). Western blot analysis shows that the levels of phosphorylated IRS1 (Serine 616; pIRS-616) are elevated in the CSF of individuals diagnosed with Alzheimer's Disease.

### Example 3: Treatment of a Human Patient With Alzheimer's Disease Having Insulin Resistance

Biological samples were obtained from two human patients with Alzheimer's Disease and two patients suspected of having Alzheimer's disease. The levels of biomarkers associated with inflammation including pIRS-636 an pIRS-616 were determined for each of the biological samples. In each of the four patients, the levels of pIRS-636 and pIRS-616 were elevated as compared to the level of these biomarkers present in patients that do not have Alzheimer's Disease. All of the patients were then treated with a therapeutically effective amount of an incretin receptor agonist (IRA).

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of diagnosing and treating a subject with Alzheimer's Disease, the method comprising:
a. obtaining a biological sample from the subject;
b. detecting one or more insulin resistance biomarkers in the biological sample;
c. determining that the subject has Alzheimer's Disease where one or more insulin resistance biomarkers are detected in the biological sample; and
d. administering to the subject a therapeutically effective amount of one or more agents for the treatment of insulin resistance.

2. The method of claim 1, wherein the biological sample is cerebral spinal fluid.

3. The method of claim 1, wherein the insulin resistance biomarkers are selected from the group consisting of: phosphorylated Ser312, phosphorylated Ser616, and phosphorylated Ser636.

4. (canceled)

5. The method of claim 1, wherein the subject is administered one or more agents for the treatment of inflammation.

6. The method of claim 5, wherein the agent is rapamycin, minocycline, thalidomide or its analogues, rosiglitazone, an NSAID, or pioglitozone.

7. The method of claim 6, wherein the NSAID is naproxen or rofecoxib.

8. The method of claim 1, wherein the subject is administered one or more agents for the treatment of oxidative stress.

9. The method of claim 8, wherein the agent is hydrogen peroxide.

10. The method of claim 1, wherein the subject is administered one or more agents for the treatment of insulin resistance.

11. The method of claim 10, wherein the agent is insulin, thiazolidinediones, an incretin receptor agonist (IRAs), glucagon like peptide 1 (GLP-1), glucagon like peptide 2 (GLP-2), or a peroxisome proliferator-activated receptor gamma (PPARγ) agonist.

12. The method of claim 11, wherein the IRA is liraglutide.

13. The method of claim 11, wherein the PPARγ agonist is rosiglitazone.

14.-38. (canceled)

39. A method for diagnosing a subject in need thereof with Alzheimer's Disease or determining susceptibility of a subject in need thereof to developing Alzheimer's Disease, the method comprising:
a. obtaining a biological sample from the subject;
b. detecting one or more biomarkers in the biological sample selected from the group consisting of: inflammation biomarkers, oxidative stress biomarkers, insulin resistance biomarkers, and autophagy biomarkers; and
c. diagnosing the subject with Alzheimer's disease where one or more of the biomarkers is detected in the biological sample or diagnosing the subject as susceptible to developing Alzheimer's Disease where one or more of the biomarkers is detected in the biological sample.

40. The method of claim 39 wherein the biological sample is cerebral spinal fluid (CSF).

41. The method of claim 39, wherein the biological sample is blood.

42. The method of claim 39, wherein the inflammation biomarker is p38 MAPK.

43. The method of claim 39, wherein the insulin resistance biomarker is insulin receptor substrate 1 (IRS-1).

44. The method of claim 43, wherein the IRS-1 is phosphorylated at Ser616.

45. The method of claim 43, wherein the IRS-1 is phosphorylated at Ser636.

46. The method of claim 43, wherein the IRS-1 is phosphorylated at Ser616 and Ser636.

47. The method of claim 39, wherein the autophagy biomarker is p62.

48. The method of claim 39, wherein the oxidative stress biomarker for is DNA damage, cleaved caspase 3, gamma-H2AX, BAD, or BAX.

49. The method of claim 39, wherein the subject is determined to have Alzheimer's Disease where the one or more of the biomarkers detected in the biological sample are present in an amount at or above a threshold.

50. The method of claim 49, wherein the threshold is the amount of the one or more biomarkers in a biological sample obtained from a subject without Alzheimer's Disease.
